(19) United States
(12) Patent Application Publication
(10) Pub. No.: US 2010/0010091 A1
(43) Pub. Date: Jan. 14, 2010

(54) Anhydrous, Dermatological or Cosmetic Preparation Containing Urea

(75) Inventor: Hans R. Suess, Starrkirch-Wil (CH)

Correspondence Address: Larson & Anderson, LLC, P.O. BOX 4928, DILLON, CO, 80435, US

(73) Assignee: DR. H.R. SUESS AG, Dulliken (CH)

(21) Appl. No.: 12/439,058

(22) PCT Filed: Aug. 20, 2007
(86) PCT No.: PCT/CH2007/000407

§ 371 Date: Feb. 26, 2009

(30) Foreign Application Priority Data
Aug. 28, 2006 (CH) 1371/06

(51) Int. Cl. A61K 31/17

(52) U.S. Cl. 514/588

(57) ABSTRACT

A dermatological or cosmetic preparation, which is anhydrous and contains urea, and is administered to improve the water retention capability of the stratum corneum, contains phytantriol and comprises a gel-like basis, which is essentially free of straight-chained hydrocarbons with 6 to 50 C-atoms. Compared with known such preparations, the preparation comprises markedly improved dermal sensory attributes.

### BACKGROUND OF THE INVENTION

The invention concerns an anhydrous dermatological or cosmetic preparation that contains urea.

In the body of mammals, urea (Carbamide, CAS No. 57-13-6) occurs as an end product of protein decomposition and does not therefore provide a source of energy for most pathogenetic microbes. Urea is a constituent of urine and has an anti-microbial effect. Presumably for this reason urine was applied to infected wounds in ancient Babylon. Urea is also contained in the stratum corneum of the skin as a component of the moisturising factor (NMF). This moisturising factor is responsible for a sufficient water content in the stratum corneum and about 7% of it is constituted by urea. If the stratum corneum contains too little urea, the water retention capacity is reduced and the trans-epidermal loss of water increases, thus rendering the skin dry, flaking and susceptible to irritation. In various skin diseases, such as psoriasis, the water content of the stratum corneum is greatly reduced, in the case of neurodermatoses (atopic dermatitis) by up to 80%. It therefore makes sense to supply dry skin directly with urea.

Urea is bipolar and therefore highly soluble in water and able to interact with ionic saline. Since urea is not soluble in lipids, it is mostly administered in emulsions, wherein o/w-emulsions (oil in water) penetrate the skin more rapidly than w/o-emulsions (water in oil). In hydrous preparations however, urea slowly disintegrates into carbon dioxide and ammonia. This process may be slowed down, albeit not entirely prevented, by storage at low temperatures and/or by adding acids, e.g. hydroxy acids such as glycolic or lactic acid. Also known are anhydrous urea containing preparations, which do not suffer the disadvantage of ureic decomposition. Although such preparations take longer than emulsions to be absorbed by the skin, they increase the moisture content of the stratum corneum more effectively. This is due to the fact that, in addition to the hydrating character of the urea, the anhydrous preparations also have an occluding effect.

Already in 1960, Arievich in Russia used an anhydrous urea containing preparation (emplastrum urea) to remove nails, which had been destroyed by fungal infection (Cutis 25:605-612, 1980). The preparation according to Arievich contains 40% urea, 25% white Vaseline, 20% lanolin USP, 5% beeswax and 10% silica gel. The urea is ground finely to avoid the preparation turning out grainy.

An anhydrous stick consisting of urea and predominantly inert lipids such as paraffin is described in the patent application GP 2 157 173 (Rhoem Pharma GmbH, Germany; 1984).

The patent application WO 02/47643 describes an anhydrous urea containing preparation, which is based on vaseline, paraffin-oil and/or microcrystalline waxes. The description of procedure mentions the importance of working as anhydrously as possible, since the urea dispersed in a hydrophobic phase often tends to re-crystallize, which provokes a distinct and unpleasant “sandpaper” sensation upon the skin and may well be the reason why such anhydrous preparations are rarely found on the market.

However, it is more likely that this “sandpaper”-effect is induced by the combination of urea and straight-chain aliphatic compounds rather than by re-crystallized urea. Friederich Bengen has already described such inclusion complexes or addition compounds in the publication DE-12438 (1940). Straight-chain aliphatic hydrocarbons with at least 6 and at most 50 C-atoms form defined complexes with urea, whereas hydrocarbons having branched structures or comprising rings usually do not. This is also shown in W. J. Zimmerschied et al., “Cristalline Adducts of Urea with Straight-chain Aliphatic Compounds”, Ind. Eng. Chem: 42:1300-1306 (1950).

It is further known that the higher the content of urea in a urea containing preparation the more likely it is to provoke skin irritations.

Thus the object of the invention is to create an anhydrous urea containing dermatological or cosmetic preparation, which, even with a comparatively low content of urea, and therefore without the aforementioned skin irritations, is capable to markedly improve the water retention capacity of the stratum corneum, but does not have the “sandpaper”-effect also mentioned above. It is in particular the object of the invention to improve the skin sensation (sensory attributes) of known anhydrous urea containing preparations without reducing the effect upon the water retention capacity of the stratum corneum. This is of particular importance because any preparation, may it be ever so effective dermatologically, cannot meet with any compliance and therefore cannot reach its potential, if it cannot offer positive sensory attributes.

### SUMMARY OF THE INVENTION

The present invention provides a dermatological or cosmetic preparation that meets this objective. The preparation is an anhydrous composition comprising urea and phytantriol. The preparation may contain a gel-like, ointment carrier.

### DETAILED DESCRIPTION OF THE INVENTION

According to the invention, the anhydrous urea containing preparation contains phytantriol (CAS-No. 74563-64-7; 3,7,11,15-tetramethylhexadecane-1,2,3-triol) as an additional active substance and advantageously comprises a gel-like ointment basis, which is essentially free of straight-chain hydrocarbons with 6 to 50 C-atoms, i.e. contains less than 5 weight percent (preferably less than 2 weight percent) of such hydrocarbons.

By adding phytantriol, which in itself increases the water retention capacity of the stratum corneum, a great effect can be achieved even with urea concentrations that are low enough not to cause any skin irritations. Because phytantriol is known to enhance the skin's ability to absorb amino acids, it may be assumed that it also enhances the ability to absorb urea, which constitutes a further advantage of the preparation according to the invention. Phytantriol further demonstrates a high solubility in lipid and due to its branched structure has no tendency to form additive compounds with the urea.

Phytantriol further improves the dermal sensory attributes by reducing the tendency of the preparation to adhere to non-dermal substances. Once applied, the preparation is less likely to be removed from the skin and thus remains on the skin for a longer time. This increases its effectiveness even further, both ecologically as well as economically.

By choosing the aforementioned ointment basis consisting advantageously of at least one liquid branched-chain, preferably aliphatic, hydrocarbon and at least one solid polymer hydrocarbon (with branched or straight chains and with more than 50 C-atoms), the aforementioned ureic complexes and therefore the “sandpaper”-effect are avoided.

The phytantriol content of the preparation according to the invention ranges from 0.1 to 10 weight percent, in particular 0.2 to 5 weight percent, preferably 2 weight percent, and the urea content ranges from 3 to 50 weight percent, in particular from 3 to 20 weight percent, preferably from 5 to 10 weight percent.

The preparation according to the invention may also comprise further micronized active ingredients such as e.g. pigments or UV-filters and/or cosmetic or dermatological agents dissolved in the liquid branched-chain hydrocarbon.

Phytantriol is a tetraisoprenoid. Isoprene is the structural unit of many natural products such as terpenes, steroids or caoutchouc. Isoprenoid constituents are also present in vitamins of group A and in chlorophyll. Phytane is found in the liver, in oil shale and other sediments, and also in meteorites (Kates, M. Biochemistry 1967, 6, 3329). The corresponding alcohol, phytanol, is a component of the human enzyme. The cell wall of the archaea consists of ethers of phytanol with glycerine, which are considerably more hydrophobic than the triglycerides of the epidermal barrier in mammals.

A liquid branched-chain hydrocarbon suitable for the ointment basis of the preparation according to the invention is e.g.: isoparaffine C11-C13 (Isopar L, Exxon), paraffin oil (Mineral Oil, CAS Nr. 80122-95-1, 8020-83-5 or 8042-47-5), hydrated polydecenes (CAS Nr. 25189-70 or 37309-58-3), isododecane (CAS Nr. 141-70-8, 13475-82-6 or 31807-55-3) isohexadecane (CAS Nr. 4390-04-9 or 60908-77-2), isoeicosane (CAS Nr. 52845-07-5) or mixtures of at least two of the aforementioned hydrocarbons.

A solid polymer hydrocarbon suitable for the ointment basis of the preparation according to the invention is e.g. polyethylene (CAS Nr. 9002-88-4), polypropylene (CAS Nr. 9003-07-0), polybutene (CAS Nr. 9003-28-5 or 9003-29-6), polyisobutene (CAS Nr. 9003-31-0), polystyrene (CAS Nr. 9003-53-06), ethylenepropylene (CAS Nr. 9010-79-1) or corresponding copolymers or mixtures of at least two of the aforementioned polymers.

The liquid branched-chain hydrocarbon is blended with the solid polymer hydrocarbon, resulting in a pasty gel.

An exemplary formulation contains 84 weight percent of Isopar L (Exxon), 9 weight percent of polyethylene (e.g. Luwax A, BASF), 5 weight percent of urea (pharm. quality) and 2 weight percent of phytantriol.

The preferred process of manufacturing the preparation according to the invention is to add the solid polymer hydrocarbon to the liquid branched-chain, preferably aliphatic hydrocarbon at a temperature of around 125° C. and to stir the mixture until it forms a clear gel. Then the micronized urea and the phytantriol are added at 110 to 115° C., i.e. at a markedly lower temperature than the melting point of urea (133° C.) consequently the mixture is cooled down to room temperature whilst being stirred throughout the process. The micronized urea can be prepared e.g. in an open pinned disk mill (e.g. type 160 by Alpine Augsburg) at ambient temperature and normal humidity without adding any dehumidifying means.

Stored at between 15 and 30° C., the preparation produced according to the aforementioned recipe remains entirely homogenous for at least a year, even if a commercially available phytantriol containing up to 0.5% water was used in the process.

### Examples

### Composition in Weight Percent

| | Preparation no. | | | | | | | |
|---|---|---|---|---|---|---|---|---|
| | 1 | | 3 | 4 | | | | |
| | ref | 2 | ref | ref | 5 | 6 | 7 | 8 |
| C11-C13 Isoparaffine | | | | | 79 | | | |
| Paraffin oil | | | | | | 79 | | |
| Polydecene | | | | | | | | 81 |
| Isododecane | 16 | 16 | 16 | 16 | | | | |
| Isohexadecane | | | | | | | 81 | |
| Isoeicosane | 65 | 65 | 65 | 65 | | | | |
| Polyethylene | 12 | 12 | 12 | 12 | 9 | 9 | 12 | 12 |
| Urea, micronized | 5 | 5 | 5 | 5 | 10 | 10 | 5 | 5 |
| Phytantriol | | 2 | | | 2 | 2 | 2 | 2 |
| Glyceryl oleate | | | 2 | | | | | |
| Glyceryl isostearate | | | | 2 | | | | |

Skin sensory tests were conducted for all preparation examples listed in the table above. The assessments of the dermal sensory attributes of the preparations were executed according to the principles of Morten Meilgaard, Gail Vance Civille and Thomas Carr (“Sensory Evaluation Techniques”, 3^{rd} Edition 1999, CRC Press, pp 184-186) and presented the following results:
Compared with preparation 1, there is a marked improvement in the sensory perception of preparation 2, differing from the reference preparation 1 merely by the content of phytantriol. The sensory perception of preparation 2 is more pleasant than the sensory perception of corresponding o/w-emulsions.
Similar to the reference preparation 1, the reference preparation 3, containing glyceryl oleata instead of phytantriol, does not result in an improvement of the sensory perception. Phytantriol, containing 0.5% water in its commercially available form, spontaneously forms a cubic crystal system in a nano-range, into which dermatological additives can be incorporated, increasing their ability to penetrate. The comparison between preparation 2 and the reference preparation 3 containing glyceryl oleate, which also forms such cubic systems, implies that the improvement of the sensory perception achieved with phytantriol cannot be attributed to these systems.
The reference preparation 4, where glyceryl isostereate is added as opposed to phytantriol in preparation 2, shows a slight improvement of the sensory perception when compared with the reference preparation 3, which is probably due to the branched structure of the isostearate.
Even the preparations with high urea contents (preparations 5 and 6: 10%) can be applied to the facial skin without any unpleasant side effects such as stinging or itching.
In preparations containing the low-viscosity isoparaffine (preparation 5), the sensory attributes improve more rapidly than in preparations containing more viscous paraffin oil, which is due to the increased gliding quality in the application to the skin and to a reduction of the fatty qualities. These results are mirrored in the comparison between preparation 7, containing low-viscosity isohexadecane, and preparation 8, containing more viscous polydecene.

1-13. (canceled)

14. An anhydrous dermatological or cosmetic preparation comprising urea and phytantriol.

15. The preparation according to claim 14, wherein the urea and phytantriol are present in an ointment base, and wherein the ointment base is essentially free of straight-chained hydrocarbons comprising 5 to 50 carbon atoms.

16. The preparation according to claim 15, wherein the ointment base comprises at least one liquid branched hydrocarbon and at least one solid polymer hydrocarbon.

17. The preparation according to claim 16, wherein the branched aliphatic hydrocarbon is isoparaffin C11-C 13, paraffin oil, a hydrated polydecene, an isododecane, an isohexadecane, an isoeicosane or a combination thereof, and that the solid polymer hydrocarbon is a polyethylene, a polypropylene, a polybutene, a polyisobutene, a polystyrene, an ethylenepropylene, a copolymer thereof or a mixture thereof.

18. The preparation according to claim 16, wherein the urea is micronized.

19. The preparation according to claim 15, wherein the urea is micronized.

20. The preparation according to claim 15, wherein the concentration of phytantriol in the preparation is between 0.1 and 10 weight percent.

21. The preparation according to claim 20, wherein the concentration of phytantriol in the preparation is between 0.2 to 3 weight percent.

22. The preparation according to claim 21, wherein the concentration of urea in the preparation is between 3 and 50 weight percent.

23. The preparation according to claim 22, wherein the concentration of urea in the preparation is between 3 and 20 weight percent.

24. The preparation according to claim 20, wherein the concentration of urea in the preparation is between 3 and 50 weight percent.

25. The preparation according to claim 24, wherein the concentration of urea in the preparation is between 3 and 20 weight percent.

26. The preparation according to claim 14, wherein the concentration of phytantriol in the preparation is between 0.1 and 10 weight percent.

27. The preparation according to claim 26, wherein the concentration of phytantriol in the preparation is between 0.2 to 3 weight percent.

28. The preparation according to claim 27, wherein the concentration of urea in the preparation is between 3 and 50 weight percent.

29. The preparation according to claim 28, wherein the concentration of urea in the preparation is between 3 and 20 weight percent.

30. The preparation according to claim 26, wherein the concentration of urea in the preparation is between 3 and 50 weight percent.

31. The preparation according to claim 30, wherein the concentration of urea in the preparation is between 3 and 20 weight percent.

32. The preparation according to claim 14, wherein the preparation further comprises one or more additional cosmetic or dermatological agents.

33. The preparation according to claim 32, wherein the preparation further comprises pigments or UV-filters as additional cosmetic or dermatological agents.

34. The preparation according to claim 14, wherein the composition comprises 16 weight percent of isodecane, 65 weight percent of isoeicosane, 12 weight percent of polyethylene, 5 weight percent of urea and 2 weight percent of phytantriol.

35. A method for improvement of the dermal sensory attributes of an anhydrous, urea-containing, dermatological or cosmetic preparation, comprising adding to the preparation an effective amount of phytantriol.

36. The method according to claim 35, wherein the dermatological or cosmetic preparation comprises urea and the phytantriol in an ointment base.

37. The method according to claim 36, wherein the preparation comprises 0.1 and 10 weight percent phytantriol.

38. The method according to claim 37, wherein the preparation comprises between 0.2 to 3 weight percent phytantriol.
